(19) United States
(12) Patent Application Publication
(10) Pub. No.: US 2002/0166558 A1
(43) Pub. Date: Nov. 14, 2002

(54) Birth control device, invented by Oliver Jack Somers-Wilder, primary inventor, and Connie Jean Somers-Wilder, both citizens of the United States and reside at 5109 Madera Terrace in Oklahoma City, Oklahoma, 73129, preventing pregnancy for extended periods of time and is quickly reversible by medical procedure

(76) Inventors: Oliver J. Somers-Wilder, Oklahoma City, OK (US); Connie Jean Somers-Wilder, Oklahoma City, OK (US)

Correspondence Address: Oliver Jack Somers-Wilder, 5109 Madera Terrace, Oklahoma City, OK, 73129, US

(21) Appl. No.: 09/852,614

(22) Filed: May 11, 2001

(51) Int. Cl. A61F 6/06

(52) U.S. Cl. 128/830

(57) ABSTRACT

This is a birth control device that prevents pregnancy for extended periods of time with the same degree of certainty as permanent sterilization: however, this sterilization is easily reversible when childbearing is desired. This device is implanted through a medical procedure. It consists of two tubes that may be positioned either in a closed or open position and held there by a clip to either prevent or allow fertilization of the egg. Accessing this device to change from open to closed or vice versa requires a medical procedure.

### CROSS-REFERENCE TO RELATED APPLICATIONS

None.

### STATEMENT REGARDING FEDERALLY SPONSORED RESEARCH OR DEVOPMENT

None.

### REFERENCE TO A MICROFICHE APPENDIX

None

### BACKGROUND OF THE INVENTION

Currently birth control can be accomplished through use of temporary devices or chemicals utilized with each episode of intercourse or daily pills. These are easily forgotten or omitted, resulting in unwanted pregnancies. Other chemicals and devices offer longer protection; however, all of these have some potential side effects or hazards to use, some of which are quit serious. Permanent sterilization has been used when no children are desired. This invention will allow permanent birth control that is easily reversible in either the male or female.

### BREIF SUMMARY OF THE INVENTION

This device may be implanted through a medical procedure in the fallopian tubes of a women or the seminal vesicles of a man in the closed position at the time sexual activity begins. This would prevent the egg or sperm from traveling to a point where fertilization could occur. When pregnancy and children are desired, the device is again accessed through a medical procedure and the device is again adjusted to the open position. In the open position, the egg and sperm would cycle and travel normally, allowing pregnancy to occur.

### BRIEF DESCRIPTION OF TH SEVERAL VEIWS OF THE DRAWING

FIG. 1-Proximal section of the tube, which includes the cube (A).

FIG. 1-A—Attached cube with one hinged side. The edges of the cube are smoothed and rounded.

FIG. 1-B—Hinged side of cube that covers tube opening when in the closed position, the top of which is equal to the flange of the proximal tube. Inside of the flap is to be covered with soft gasket material to facilitate the tight seal.

FIGS. 1 & 2-C—Soft “O” ring to seal closure.

FIGS. 1 & 2-D—Flanged area behind “O” ring to support clip in place.

FIG. 1-E—Additional open side of cube when hinged piece (B) is down in the closed position. They allow egg or sperm to be reabsorbed, as they cannot get through the closed tube.

FIG. 1-G1—Points where the clip passes through, behind the flanges when tubes are in the open position.

FIG. 1-G2—Points where the clip passes over the hinged piece and behind the flange when in the closed position.

FIG. 1-H—Three open sides of cube to allow reabsorption of the egg or sperm when they cannot get through the closed tube.

FIG. 2—Distal section of the tube.

FIG. 3—Two views of the colp, which will hold the two sections of tube together. It will also keep the hinged side in place in the closed position when set in this position.

### DETAILED DESCRIPTION OF THE INVENTION

This is an implanted device for birth control that is quickly reversible through a medical procedure. The device consists of two tubes that are of a size that they may be place in the fallopian tubes of a woman or seminal vesicles of a man.

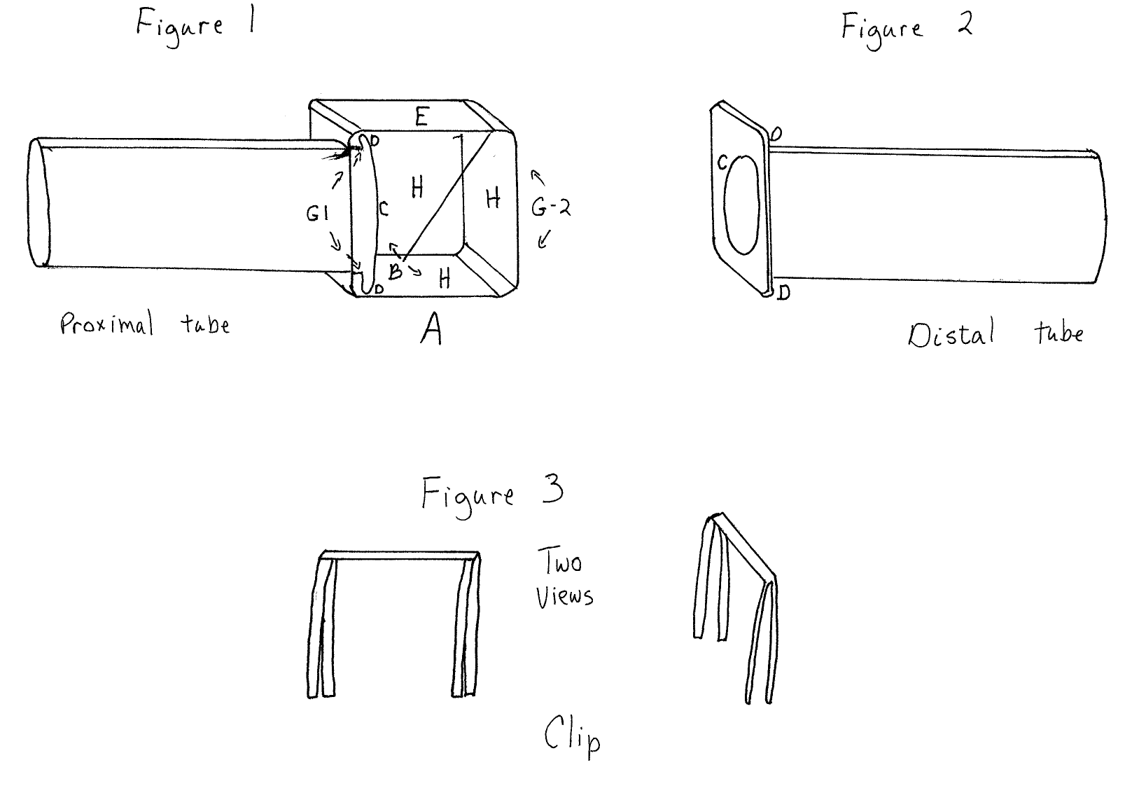

The device consists of two tubes (FIGS. 1 & 2 whose joining ends are closed in a cube with three open sides. The cube is rounded on all edges and corners to make it “body friendly.” (See A in FIG. 1).

The proximal tube (FIG. 1) is so named as it is placed closest to the point of origin of the egg, the ovary. In the male, it is placed in the seminal vesicles, just above the juncture with the urethra, to prevent their exit. The distal tube (FIG. 2) is, of course, further from the point of origin and is joined to the proximal when implanted.

Prior to implantation of the device, the two tubes (FIGS. 1 & 2 are brought together with the hinged side (FIG. 1-B) of the cube down over the opening of the distal portion. It is held in place by the clip inserted at the points indicated as G2. The “O” ring (FIG. 2-C), will form a seal, being held tightly against the hinged side (FIG. 1-B) by the clip (FIG. 3). The clip is inserted through an open side (H) and goes over the hinged side of the cube and the flanged areas (FIGS. 1-B & 2-D). When the tube is “closed,” the egg or sperm can be reabsorbed through the open sides of the cube (FIGS. 1-E & H).

During the time when pregnancy is desired, this process is reversed by a medical procedure that removes the clip to raise the hinged section (FIG. 1-B) by the clip (FIG. 3). The clip is inserted through a non-hinged side of the cube in the G2 position, behind the flanged areas (FIGS. 1-D & 2-D). This is the medial (joining) end of the tubes. When the tube is “closed,” the egg or sperm can be reabsorbed through the open sides of the cube (FIGS. 1-E & H). The hinged section will rest on the outside of the distal tube (FIG. 2) and essentially close the side of the cube (FIG. 1-E) so that the egg and sperm will cycle in the normal manner, allowing conception.

When desired childbearing years are completed, the device would be accessed to close the device as previously described. If childbearing had occurred before the device was implanted, this could be the first and only procedure, yet it leaves the option later for more children.

The materials to manufacture this product will be biologically compatible with the human body, a rigid and semi-rigid nylon type of material. The non-connected ends of both tubes will be semi-rigid materials that can be surgically attached to the fallopian tubes or seminal vesicles.

1. What we claim as our invention is the unique combination of these structures, the tubes, cube, hinged side, clip, and the quick open-close feature, with their unique use in birth control. This utilized a combination of plumbing and medical knowledge for design and technique.
